(19) United States
(12) Patent Application Publication
(10) Pub. No.: US 2002/0002375 A1
(43) Pub. Date: Jan. 3, 2002

(54) Utensil for extracting brain matter from cattle

(76) Inventors: Thierry Legeai, Boos (FR); Maurice Venturini, Dieppe (FR)

Correspondence Address: YOUNG & THOMPSON, 745 SOUTH 23RD STREET 2ND FLOOR, ARLINGTON, VA, 22202

(21) Appl. No.: 09/769,247

(22) Filed: Jan. 26, 2001

(30) Foreign Application Priority Data
Jun. 30, 2000 (EP) 00401879.2

(51) Int. Cl. A61B 17/32

(52) U.S. Cl. 606/167

(57) ABSTRACT

The invention provides a utensil (10) for extracting a sample of cerebral matter, particularly the brain stem from cattle, for laboratory analysis purposes, characterized in that it comprises a manipulation handle (12), at the front end of which is arranged a hollow part forming an extraction spoon (14), and the peripheral edge of which comprises at least one cutting portion (22, 26, 30) for cutting into the sample prior to its extraction.

The present invention relates to a utensil for extracting a sample of viscera for laboratory, medical or veterinary analysis purposes.

The invention relates in particular to a utensil for extracting the brain stem located in the cranial cavity of cattle.

In order to pursue an effective health policy, it has been found necessary to be able to carry out massive campaigns involving taking samples of brain tissue from cattle in order to carry out veterinary analysis to determine whether the animal from which the sample has been taken is affected by a disease such as Bovine Spungiform Encephalopathy or BSE.

In the case of cattle, the part concerned consists, for example, of the brain stem located in the skull of the cattle.

In order to access this brain stem which is in the continuation of the spinal cord, it is necessary to access the rear part of the base of the skull of the cattle, then to uncover the occiputal orifice through which access can be had to the brain case.

In this situation, it is necessary to be able to take the sample as accurately as possible, given the number of samples to be taken, while at the same time avoiding damaging the sample before the analysis operations, and also observing hygiene and cleanliness requirements.

The extraction operation has traditionally been performed using a scalpel or similar cutting tool that allows the brain stem to be cut, and more precisely detached and extracted from the cranial cavity.

Indeed the brain stem forms part of the encephalon and constitutes the caudal and ventral part of this organ. It is linked with the spinal cord at the rear and with the cerebellum and cerebral hemispheres at the front. Most of the roots of the cranial nerves emerge laterally from it.

The operation which consists in detaching the brain stem is therefore an operation which is complicated to perform using a scalpel and requires great skill on the part of the person taking the sample.

Once the cutting has been performed, the extract or sample then has to be collected under the conditions mentioned above.

The extraction operation therefore entails, overall, according to the state of the art, the use of at least two tools, one for cutting and the other for extracting in order to collect the sample and is particularly complicated to perform.

In the case of each tool—cutting and extracting—it is also necessary to clean these and sterilize them between each extraction operation in order not to contaminate one sample with remains of the sample extracted previously and which are deposited on the tools.

Given the above, it has been found desirable to be able to have a new tool for taking such samples which overcomes the drawbacks which have just been mentioned and which is preferably of a low cost so that it can be disposable, that is to say so that a separate tool can be used for each extraction.

To this end, the invention provides a utensil for extracting a sample of cerebral matter, particularly from the brain stem located in the cranial cavity of cattle, for laboratory, medical or veterinary analysis purposes, characterized in that it comprises a manipulation handle, at the front end of which is arranged a hollow part forming an extraction spoon, and the peripheral edge of which comprises at least one cutting portion for cutting into the sample prior to its extraction.

By virtue of the design according to the invention, there is thus available a single utensil or tool which allows the operation of taking the sample to be performed ergonomically and easily.

Its particularly simple design allows it to be produced very economically, for example in a single piece moulded of plastic, and it may thus be a one-use utensil, that is to say that the utensil can be produced at a very low cost and disposed of after the extraction operation, or may alternatively constitute a temporary means of transferring the sample contained in the extraction spoon.

According to other features of the invention:

the peripheral edge of the spoon comprises two longitudinal portions parallel to the overall axis of the utensil and produced in the form of cutting portions;

the peripheral edge of the spoon comprises a transverse free end portion roughly perpendicular to the overall axis of the utensil and produced in the form of a cutting portion;

the transverse cutting free end portion has sawteeth;

the longitudinal portions of the edge of the spoon are connected to the transverse free end portion by connecting portions produced in the form of cutting portions;

each cutting portion is produced by virtue of a reduction in the thickness of the spoon near its peripheral edge so as to produce a cutting filament;

the concave side of the spoon has at least one projection intended to hold the sample in the spoon while it is being extracted;

each projection has an inclined front face to make it easier for the utensil to be introduced forwards, and a transverse retaining rear face which runs in a plane perpendicular to the longitudinal axis of the spoon;

in cross section on a plane perpendicular to the longitudinal axis of the spoon, the concave face thereof has a profile in the shape of an arc of a circle.

Other features and advantages of the invention will become apparent from reading the detailed description which will follow, for an understanding of which reference will be made to the appended drawings, among which:

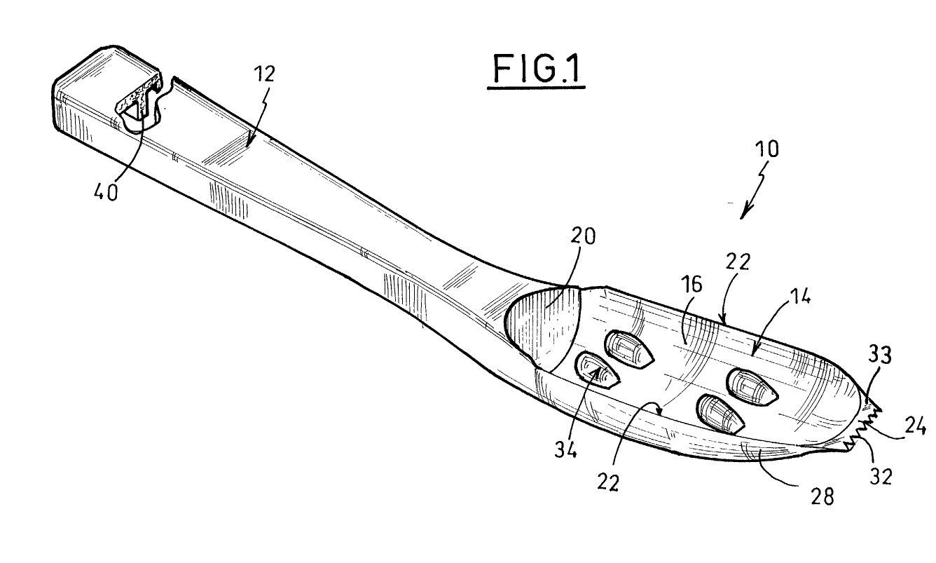

FIG. 1 is a perspective view of the utensil according to the invention;

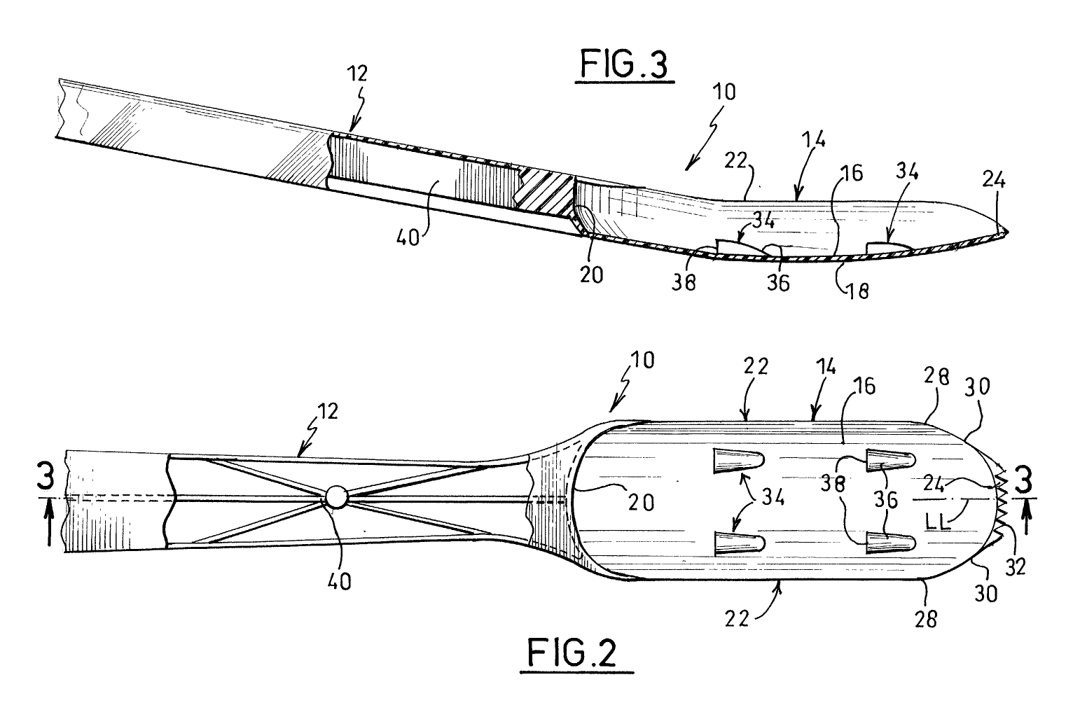

FIG. 2 is a view from above of the utensil of FIG. 1;

FIG. 3 is a view in longitudinal section on 3-3 of FIG. 2.

In the embodiment depicted in the figures, the utensil has overall symmetry of design with respect to a longitudinal vertical mid-plane passing through its longitudinal axis L-L.

In order to make the description and claims easier to understand, we will use a vertical orientation of the spoon, that is to say that the spoon will extend in a horizontal overall plane with its concave side facing vertically upwards while its roughly parallel convex side faces vertically downwards.

The utensil 10 consists mainly of a manipulation handle 12 the overall shape of which is elongate along a central longitudinal axis L-L.

The figures depict a utensil 10 made in accordance with the teachings of the invention and which is more particularly suited here to the extraction of the brain stem from the cranial cavity of cattle.

The handle 12 is continued beyond its front end in the form of an extraction spoon 14.

The overall shape of the spoon is elongate along the axis L-L and the profile is in the shape of an arc of a cylinder, vertically delimited at the top by a concave side 16 and vertically delimited at the bottom by a convex side 18.

The spoon 14 is delimited longitudinally towards the rear end by a transverse rear face 20 facing generally vertically and with a slightly rounded profile as can be seen in particular in FIG. 1.

The concave profile 16 of the spoon is, in section on a transverse vertical plane perpendicular to the axis L-L, a profile in the shape of an arc of a circle which is roughly constant right along the spoon, so as to give the latter a profile which overall is in the shape of an arc of a cylinder.

The spoon is laterally delimited by two parallel and opposite longitudinal edges 22 and is delimited longitudinally towards the front end by a transverse free end front edge 24, the opposite transverse ends of which are connected to the front longitudinal ends 28 of the longitudinal edges 22 by two connecting portions 30.

The longitudinal edges 22 are parallel straight edges while the front transverse edge 24 is itself also approximately straight and perpendicular to the axis L-L while the connecting portions 30 may be straight or slightly curved so as to make the connection between the edges 22 and 24.

According to an essential feature of the invention, the various longitudinal portions 22 and transverse portions 24 of the overall peripheral edge of the spoon 14 are made in the form of cutting portions allowing the cutting operations to be performed by manipulating the utensil 10.

Each cutting edge or portion is preferably made by virtue of a thinning of material, that is to say by virtue of a reduced thickness of the spoon near its peripheral edge by comparison with the average thickness of the spoon between its concave side 16 and convex side 18.

The front free end transverse edge 24 is a cutting portion and may also have teeth 32 which extend longitudinally forward to constitute a cutting and toothed edge in the manner of a toothed cutter, also known as a serrated cutter.

In order to produce a cutting filament more easily, particularly in the case of the front free end portion 24, it may be desirable to form a small flat 33, whose intersection with the external envelope of the convex bottom side 18 defines the cutting edge.

In the embodiment depicted in the figures, the concave internal side 16 of the extraction spoon 14 has four identical projections or studs 34 for retaining the sample taken.

The four projections are in this instance arranged in the form of two pairs of projections aligned longitudinally one behind the other.

Each projection formed in relief or projecting vertically upwards in the concave side 16 has a front face 36 which, overall, is inclined with respect to the plane at the bottom of the spoon and a transverse rear retaining face 38 oriented vertically and perpendicular to the axis L-L.

As a preference, particularly to make the spoon easier to manufacture in a moulding operation of plastic in a single piece, the front face 36 of each projection 34 is in the form of a rounded and inclined boss.

The design according to the invention, in which the spoon 14 is located roughly in the longitudinal continuation of the handle 12, possibly making a small angle of about 10° with respect to the latter, makes it possible for the utensil 10 to be produced easily by moulding of plastic in a single operation, the moulding operation also making it possible to produce a handle 12 which is hollow, with strengthening ribs 40.

In the case of a utensil for extracting the brain stem from cattle, the transverse width of the spoon, that is to say the distance separating its two longitudinal edges or portions 22 is about 30 millimetres, while the length of the spoon is about 80 millimetres.

In the case of the extraction mentioned earlier in cattle, the utensil just described is used as follows.

Having uncovered access to the occiputal cavity, the user, for example a veterinary surgeon, introduces the spoon longitudinally forwards, preferably holding it upside down, that is to say with its concave side facing downwards, that is to say towards the ventral part of the encephalon.

In order to perform correct and sufficient extraction, the spoon needs to be guided, with the convex side uppermost, in a gradual forwards movement along the axis of the medulla oblongata, from the occiputal hole and under the dura mater.

The cerebral cavity is entered. The anterior edge of the spoon cuts the base of the cerebellum, isolating the cerebellar peduncles (anterior, middle and posterior) using the toothed cutting edge 24, 32.

By rotating the spoon from left to right and from right to left, the roots of the cranial nerves are severed using the cutting edges 22.

Thus isolated, the brain stem is cut off at the rear of the quadrigeminal bodies using a movement involving tilting the tip of the spoon obliquely downwards and backwards and using the serrated edges 24, 32.

There is then the matter of severing the neck of the medulla oblongata in order to definitively isolate the extract, using the serrated edge 24, 32.

In doing this, the entire sample can be extracted from the cerebral cavity through the occiputal hole.

Likewise, as the spoon is introduced, its front transverse cutting edge 24, 32 plays a part in the cutting operation.

The various cutting edges allow the sample to be detached from the adjacent spinal cord and make it possible to sever the nerves or nerve endings.

Once these cutting and isolation operations have been completed, the user can introduce the spoon longitudinally once again, this time the right way up, to collect the annular protuberance and extract it from the cavity.

During introduction, the projecting projections or bosses 34 do not impair insertion because their front face 36 is inclined, whereas they do play a part in retaining the sample to allow it to be extracted rearwards, by virtue of their vertical rear transverse face 38.

The utensil according to the invention, as has just been described, therefore makes it possible to perform the operations of taking samples of viscera without having to resort to any additional cutting or severing tool, and thereafter makes the sample easier to manipulate in so far as it can be produced at low cost in the form of a disposable object.

Of course the invention is not restricted to the extraction of brain stem from cattle, but can, for example, also be used, by adapting the dimensions of the product to suit, for extracting bone marrow from inside a long bone in the medullary cavity thereof.

1. Utensil (10) for extracting a sample of viscera, particularly cerebral matter such as the brain stem located in the cranial cavity of cattle, for laboratory analysis purposes, characterized in that it comprises a manipulation handle (12), at the front end of which is arranged a hollow part forming an extraction spoon (14), and the peripheral edge of which comprises at least one cutting portion (22, 26, 30) for cutting into the sample prior to its extraction.

2. Utensil according to the preceding claim, characterized in that the peripheral edge of the spoon (14) comprises two longitudinal portions (22) parallel to the overall axis of the utensil and produced in the form of cutting portions.

3. Utensil according to any one of the preceding claims, characterized in that the peripheral edge of the spoon (14) comprises a transverse free end portion (26) roughly perpendicular to the overall axis (L-L) of the utensil (10) and produced in the form of a cutting portion.

4. Utensil according to the preceding claim, characterized in that the transverse cutting free end portion (26) has sawteeth (30).

5. Utensil according to either of claims 3 and 4 taken in combination with claim 2, characterized in that the longitudinal portions (22) of the edge of the spoon (14) are connected to the transverse free end portion (26) by connecting portions (30) produced in the form of cutting portions.

6. Utensil according to any one of the preceding claims, characterized in that each cutting portion (22, 26, 30) is produced by virtue of a reduction in the thickness of the spoon (140) near its peripheral edge so as to produce a cutting filament.

7. Utensil according to any one of the preceding claims, characterized in that the concave side (16) of the spoon has at least one projection (34) intended to hold the sample in the spoon (14) while it is being extracted.

8. Utensil according to the preceding claim, characterized in that each projection (34) has an inclined front face (36) to make it easier for the utensil to be introduced forwards, and a transverse retaining rear face (38) which runs in a plane perpendicular to the longitudinal axis (L-L) of the spoon (14).

9. Utensil according to any one of the preceding claims, characterized in that, in cross section on a plane perpendicular to the longitudinal axis of the spoon (14), the concave face (16) thereof has a profile in the shape of an arc of a circle.

10. Utensil according to any one of the preceding claims, characterized in that it is made as a single piece by moulding of plastic.
